SEQUENCE LISTING (8 sequences)

SEQ ID NO: 1 - DNA, 1410 bp, Homo sapiens
atgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaaggcaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcggctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattga

SEQ ID NO: 2 - DNA, 1410 bp, Artificial Sequence
  Synthetic
atgcactctttcccgccgctgctgctgctgctgttctggggtgttgtttctcactctttcccggctaccctggaaacccaggaacaggacgttgacctggttcagaaatacctggaaaaatactacaacctgaaaaacgacggtcgtcaggttgaaaaacgtcgtaactctggtccggttgttgaaaaactgaaacagatgcaggaattcttcggtctgaaagttaccggtaaaccggacgctgaaaccctgaaagttatgaaacagccgcgttgcggtgttccggacgttgctcagttcgttctgaccgaaggtaacccgcgttgggaacagacccacctgacctaccgtatcgaaaactacaccccggacctgccgcgtgctgacgttgaccacgctatcgaaaaagctttccagctgtggtctaacgttaccccgctgaccttcaccaaagtttctgaaggtcaggctgacatcatgatctctttcgttcgtggtgaccaccgtgacaactctccgttcgacggtccgggtggtaacctggctcacgctttccagccgggtccgggtatcggtggtgacgctcacttcgacgaagacgaacgttggaccaacaacttccgtgaatacaacctgcaccgtgttgctgctcacgaactgggtcactctctgggtctgtctcactctaccgacatcggtgctctgatgtacccgtcttacaccttctctggtgacgttcagctggctcaggacgacatcgacggtatccaggctatctacggtcgttctcagaacccggttcagccgatcggtccgcagaccccgaaagcttgcgactctaaactgaccttcgacgctatcaccaccatccgtggtgaagttatgttcttcaaagaccgtttctacatgcgtaccaacccgttctacccggaagttgaactgaacttcatctctgttttctggccgcagctgccgaacggtctggaagctgcttacgaattcgctgaccgtgacgaagttcgtttcttcaaaggtaacaaatactgggctgttcagggtcagaacgttctgcacggttacccgaaagacatctactcttctttcggtttcccgcgtaccgttaaacacatcgacgctgctctgtctgaagaaaacaccggtaaaacctacttcttcgttgctaacaaatactggcgttacgacgaatacaaacgttctatggacccgggttacccgaaaatgatcgctcacgacttcccgggtatcggtcacaaagttgacgctgttttcatgaaagacggtttcttctacttcttccacggtacccgtcagtacaaattcgacccgaaaaccaaacgtatcctgaccctgcagaaagctaactcttggttcaactgccgtaaaaactaa

SEQ ID NO: 3 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 4 - DNA, 2124 bp, Homo sapiens
atgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactaa

SEQ ID NO: 5 - DNA, 2124 bp, Artificial Sequence
  Synthetic
atgtctctgtggcagccgctggttctggttctgctggttctgggttgctgcttcgctgctccgcgtcagcgtcagtctaccctggttctgttcccgggtgacctgcgtaccaacctgaccgaccgtcagctggctgaagaatacctgtaccgttacggttacacccgtgttgctgaaatgcgtggtgaatctaaatctctgggtccggctctgctgctgctgcagaaacagctgtctctgccggaaaccggtgaactggactctgctaccctgaaagctatgcgtaccccgcgttgcggtgttccggacctgggtcgtttccagaccttcgaaggtgacctgaaatggcaccaccacaacatcacctactggatccagaactactctgaagacctgccgcgtgctgttatcgacgacgctttcgctcgtgctttcgctctgtggtctgctgttaccccgctgaccttcacccgtgtttactctcgtgacgctgacatcgttatccagttcggtgttgctgaacacggtgacggttacccgttcgacggtaaagacggtctgctggctcacgctttcccgccgggtccgggtatccagggtgacgctcacttcgacgacgacgaactgtggtctctgggtaaaggtgttgttgttccgacccgtttcggtaacgctgacggtgctgcttgccacttcccgttcatcttcgaaggtcgttcttactctgcttgcaccaccgacggtcgttctgacggtctgccgtggtgctctaccaccgctaactacgacaccgacgaccgtttcggtttctgcccgtctgaacgtctgtacacccaggacggtaacgctgacggtaaaccgtgccagttcccgttcatcttccagggtcagtcttactctgcttgcaccaccgacggtcgttctgacggttaccgttggtgcgctaccaccgctaactacgaccgtgacaaactgttcggtttctgcccgacccgtgctgactctaccgttatgggtggtaactctgctggtgaactgtgcgttttcccgttcaccttcctgggtaaagaatactctacctgcacctctgaaggtcgtggtgacggtcgtctgtggtgcgctaccacctctaacttcgactctgacaaaaaatggggtttctgcccggaccagggttactctctgttcctggttgctgctcacgaattcggtcacgctctgggtctggaccactcttctgttccggaagctctgatgtacccgatgtaccgtttcaccgaaggtccgccgctgcacaaagacgacgttaacggtatccgtcacctgtacggtccgcgtccggaaccggaaccgcgtccgccgaccaccaccaccccgcagccgaccgctccgccgaccgtttgcccgaccggtccgccgaccgttcacccgtctgaacgtccgaccgctggtccgaccggtccgccgtctgctggtccgaccggtccgccgaccgctggtccgtctaccgctaccaccgttccgctgtctccggttgacgacgcttgcaacgttaacatcttcgacgctatcgctgaaatcggtaaccagctgtacctgttcaaagacggtaaatactggcgtttctctgaaggtcgtggttctcgtccgcagggtccgttcctgatcgctgacaaatggccggctctgccgcgtaaactggactctgttttcgaagaacgtctgtctaaaaaactgttcttcttctctggtcgtcaggtttgggtttacaccggtgcttctgttctgggtccgcgtcgtctggacaaactgggtctgggtgctgacgttgctcaggttaccggtgctctgcgttctggtcgtggtaaaatgctgctgttctctggtcgtcgtctgtggcgtttcgacgttaaagctcagatggttgacccgcgttctgcttctgaagttgaccgtatgttcccgggtgttccgctggacacccacgacgttttccagtaccgtgaaaaagcttacttctgccaggaccgtttctactggcgtgtttcttctcgttctgaactgaaccaggttgaccaggttggttacgttacctacgacatcctgcagtgcccggaagactaa

SEQ ID NO: 6 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 7 - DNA, 2124 bp, Artificial Sequence
  Synthetic
atgtctctgtggcagccgctggttctggttctgctggttctgggttgctgcttcgctgctccgcgtcagcgtcagtctaccctggttctgttcccgggtgacctgcgtaccaacctgaccgaccgtcagctggctgaagaatacctgtaccgttacggttacacccgtgttgctgaaatgcgtggtgaatctaaatctctgggtccggctctgctgctgctgcagaaacagctgtctctgccggaaaccggtgaactggactctgctaccctgaaagctatgcgtaccccgcgttgcggtgttccggacctgggtcgtttccagaccttcgaaggtgacctgaaatggcaccaccacaacatcacctactggatccagaactactctgaagacctgccgcgtgctgttatcgacgacgctttcgctcgtgctttcgctctgtggtctgctgttaccccgctgaccttcacccgtgtttactctcgtgacgctgacatcgttatccagttcggtgttgctgaacacggtgacggttacccgttcgacggtaaagacggtctgctggctcacgctttcccgccgggtccgggtatccagggtgacgctcacttcgacgacgacgaactgtggtctctgggtaaaggtgttgttgttccgacccgtttcggtaacgctgacggtgctgcttgccacttcccgttcatcttcgaaggtcgttcttactctgcttgcaccaccgacggtcgttctgacggtctgccgtggtgctctaccaccgctaactacgacaccgacgaccgtttcggtttctgcccgtctgaacgtctgtacacccaggacggtaacgctgacggtaaaccgtgccagttcccgttcatcttccagggtcagtcttactctgcttgcaccaccgacggtcgttctgacggttaccgttggtgcgctaccaccgctaactacgaccgtgacaaactgttcggtttctgcccgacccgtgctgactctaccgttatgggtggtaactctgctggtgaactgtgcgttttcccgttcaccttcctgggtaaagaatactctacctgcacctctgaaggtcgtggtgacggtcgtctgtggtgcgctaccacctctaacttcgactctgacaaaaaatggggtttctgcccggaccagggttactctctgttcctggttgctgctcaccaattcggtcacgctctgggtctggaccactcttctgttccggaagctctgatgtacccgatgtaccgtttcaccgaaggtccgccgctgcacaaagacgacgttaacggtatccgtcacctgtacggtccgcgtccggaaccggaaccgcgtccgccgaccaccaccaccccgcagccgaccgctccgccgaccgtttgcccgaccggtccgccgaccgttcacccgtctgaacgtccgaccgctggtccgaccggtccgccgtctgctggtccgaccggtccgccgaccgctggtccgtctaccgctaccaccgttccgctgtctccggttgacgacgcttgcaacgttaacatcttcgacgctatcgctgaaatcggtaaccagctgtacctgttcaaagacggtaaatactggcgtttctctgaaggtcgtggttctcgtccgcagggtccgttcctgatcgctgacaaatggccggctctgccgcgtaaactggactctgttttcgaagaacgtctgtctaaaaaactgttcttcttctctggtcgtcaggtttgggtttacaccggtgcttctgttctgggtccgcgtcgtctggacaaactgggtctgggtgctgacgttgctcaggttaccggtgctctgcgttctggtcgtggtaaaatgctgctgttctctggtcgtcgtctgtggcgtttcgacgttaaagctcagatggttgacccgcgttctgcttctgaagttgaccgtatgttcccgggtgttccgctggacacccacgacgttttccagtaccgtgaaaaagcttacttctgccaggaccgtttctactggcgtgtttcttctcgttctgaactgaaccaggttgaccaggttggttacgttacctacgacatcctgcagtgcccggaagactaa

SEQ ID NO: 8 - protein, 707 aa, Artificial Sequence
  Synthetic
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHQFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

(19) United States
(12) Patent Application Publication
(10) Pub. No.: US 2019/0233808 A1
(43) Pub. Date: Aug. 1, 2019

(54) PURIFICATION OF ACTIVE SOLUBLE RECOMBINANT MATRIX METALLOPROTEINASE IN ESCHERICHIA COLI

(71) Applicants: Lokender Kumar, Golden, CO (US); Susanta K. Sarkar, Highlands Ranch, CO (US)

(72) Inventors: Lokender Kumar, Golden, CO (US); Susanta K. Sarkar, Highlands Ranch, CO (US)

(21) Appl. No.: 16/265,600

(22) Filed: Feb. 1, 2019

Related U.S. Application Data
(60) Provisional application No. 62/625,141, filed on Feb. 1, 2018.

(51) Int. Cl. C12N 9/76

(52) U.S. Cl. CPC C12N 9/6427; C12Y 304/21004

(57) ABSTRACT

A method for purifying activated human MMP in E. coli without the use of urea or APMA is provided. In the method, a non-ionic detergent is used in a lysis buffer to solubilize MMP, and the protease activities of trypsin and MMP are utilized to digest the E. coli proteins and activate pro-MMP 1.

### CROSS REFERENCE TO RELATED APPLICATION

This application claims priority and the benefit under 35 U.S.C. § 119(e) to U.S. Provisional Patent Application Ser. No. 62/625,141 filed Feb. 1, 2018, which is incorporated herein in its entirety by reference.

### REFERENCE TO SEQUENCE LISTING

This application contains a Sequence Listing submitted as an electronic text file named “4843-157_Sequence_Listing_ST25.txt”, having a size in bytes of 29 kb, and created on Feb. 1, 2019. The information contained in this electronic file is hereby incorporated by reference in its entirety pursuant to 37 CFR § 1.52(e)(5).

### FIELD OF THE INVENTION

The present invention relates to improved methods for purifying activated proteins, such as human matrix metalloproteinase proteins (MMPs), without the use of urea or 4-amino-phenylmercuric acetate (APMA), and particularly to methods that utilize proteases to digest cellular proteins and activate pro-forms of a protein, such as pro-MMP.

### BACKGROUND OF THE INVENTION

Matrix metalloproteinase proteins (also referred to as matrix metalloproteases) (MMPs) are a genus of 25 calcium- and zinc-dependent endopeptidases that are secreted in pro form as zymogens by cells. MMPs play a key role in both healthy and pathological tissue remodeling and have been linked to many diseases, including arthritis, cardiovascular disease, fibrosis, atherosclerosis, and cancer metastasis. Of particular interest are collagenase MMP1, and MMP9. MMP1 degrades the most abundant and proteolytically resistant type-1 collagen by cleaving triple-helical monomers into ¾ (TC^{A}) and ¼ (TC^{B}) fragments. MMP1 consists of three domains: a C-terminal hemopexin-like (HPX) domain, an N-terminal catalytic (CAT) domain, and a linker region. MMP s role in collagen degradation has been the subject of extensive biochemical and clinical research for many years and has motivated both the use of MMP1 itself as a bacterial collagenase in wound healing and other applications and the use of MMP1 inhibitors to prevent cancer metastasis. MMP9 is known to activate IL-10, cleave several chemokines, and is involved in neutrophil migration across the basement membrane.

Pro-MMPs have been purified in both native and recombinant forms from a variety of sources, including fibroblasts, NS0 mouse myeloma cells, Pichia pastoris, and E. coli. While recombinant protein expression in bacterial cells, such as E. coli. is in many cases efficient and inexpensive and has been used to express recombinant human proteins, many human proteins form insoluble inclusion bodies (biologically inactive aggregates of partially folded or misfolded protein molecules) when expressed in high concentrations in bacterial cells (e.g., E. coli). In an effort to reduce the losses of recombinant proteins to inclusion bodies, a combination of denaturation with urea or guanidine hydrochloride, slow refolding, and/or chromatography is often used to purify recombinant proteins, such as human MMP1, in E. coli. The pro-matrix metalloproteinase (MMP) can then be activated by cleaving parts of the N-terminal domain by any of several reagents, including APMA, plasmin, chymotrypsin, and trypsin. As a result, the purification of recombinant human MMP1 expressed in E. coli can be expensive and result in low yields of protein.

There is thus a need in the art for methods for producing and purifying human MMP proteins in E. coli that is inexpensive and yields larger quantities of protein than previous methods. It is further advantageous for such methods to avoid the use of potentially damaging reagents, such as urea and APMA.

### SUMMARY OF THE INVENTION

The present invention provides a method of purifying a protein of interest (POI), the method comprising culturing a recombinant cell comprising an expression vector that comprises a nucleic acid sequence encoding the protein of interest, lysing the cultured cells in the presence of a non-ionic detergent, contacting the soluble portion of the lysate with a protease to which the protein of interest is resistant, contacting the soluble portion of the lysate with a filter to produce a filtrate, and a retentate containing the protein off interest, thereby purifying the protein of interest.

In certain aspects, the protein of interest may be a matrix metalloproteinase (MMP). The MMP may be MMP1, MMP2, MMP3, MMP7, MMP8, MMP9, or MMP10. In certain aspects the MMP may be MMP1. In certain aspects, the MMP may be MMP9.

In certain aspects, the insoluble portion of the lysate may be physically separated from the soluble portion of the lysate. Separation of the soluble and insoluble portions of the lysate may comprise centrifugation or filtration.

In certain aspects, the cells may be bacterial cells. In certain aspects, the cells may be E. coli cells.

In certain aspects, the expression vector is capable of replicating in the cells. In certain aspects, the nucleic acid sequence may be functionally linked to a promoter. The promoter may be an inducible promoter, and may be selected from the group consisting of a T7 promoter, a trp promoter, a lac promoter, and an SP6 promoter.

In certain aspects, the expression vector may be a bacterial expression vector. In certain aspects, the expression vector may be a pET plasmid.

In certain aspects, the nucleic acid sequence encoding the POI (e.g., an MMP) may be modified to increase the amount of the POI produced, relative to the amount of the POI produced from an unmodified POI-encoding nucleic acid sequence. Modification of the nucleic acid sequence may comprise introducing silent, substitution mutations into one or more rare codons in the nucleic acid sequence, thereby eliminating one or more rare codons from the nucleic acid sequence.

In certain aspects, the non-ionic detergent may be a polyoxyethylene. In certain aspects, the non-ionic detergent may be selected from the group consisting of Tween®, Triton®, and Nonidet™-P40. The non-ionic detergent may be Triton® (polyethylene glycol p-(1,1,3,3-tetramethylbutyl)-phenyl ether). In certain aspects, the non-ionic detergent may be Triton® X-100.

In certain aspects, the step of lysing the cells may comprise sonification, sheer stress, decompression, and/or chemical lysis (e.g., lysozyme). In certain aspects, the serine protease may be trypsin, chymotrypsin, trypsinogen or pepsinogen. In certain aspects, the soluble portion of the lysate may be filtered using a filter having a MW cutoff of about 30 kDa. Filtration may be performed at a temperature less than room temperature. Filtration may be performed at less than 12° C. Filtration may be performed at a temperature in the range of about 4° C. to about 10° C.

One aspect of the disclosure is a method of purifying matrix metalloproteinase-1 protein or a matrix metalloproteinase-9 protein. The method includes providing an expression vector comprising an optimized nucleic acid sequence encoding a MMP protein. The expression vector is transformed into E. coli cells, and the E. coli cells cultured to produce the MMP protein. These cultured E. coli cells are lysed in the presence of a non-ionic detergent to produce a lysed E. coli. The lysed E. coli cells are centrifuged to form a supernatant. The supernatant is incubated in the presence of trypsin and filtered.

One aspect of the disclosure is a method of purifying a matrix metalloproteinase protein. The method includes culturing recombinant cells comprising an expression vector comprising a nucleic acid sequence encoding the MMP protein. The cells are lysed in the presence of a non-ionic detergent to produce a lysate. The lysate comprises a soluble portion and an insoluble portion. The soluble portion of the lysate is contacted with a serine protease to produce a mixture. The mixture is filtered to produce a retentate and a MMP containing filtrate to produce a purified MMP protein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the sequence of cDNA (SEQ ID NO:1) encoding MMP1. Rare codons (relative to E. coli) are bolded and underlined.

FIG. 2 shows the sequence of MMP1 encoding DNA that has been optimized for expression in E. coli (SEQ ID NO:2).

FIG. 3 shows the amino acid sequence of the MMP-1 protein (SEQ ID NO:3). Sites of activation are bolded an underlined. The R—N bond is broken by trypsin at low concentration, and the F—V bond is broken at high concentration. The T-L bond is broken by using plasmin or plasma kallikrein. The G-P bond is broken by using MMP3. The active site HEL (bold and underlined residues) is shown and can be mutated to HQL¹ and HAL² to make MMP1 catalytically inactive.

FIGS. 4A-E illustrate expression and activation of MMP1 using the optimized sequence. (4A) SDS PAGE of lysates showing expression of MMP1. Molecular weight (MW) markers (lane 1), supernatant of bacterial cell lysates without IPTG induction (lane 2, indicated by the black arrow), and with 1 mM IPTG induction (lane 3, indicated by the black arrow). (4B) Western blot of bacterial cell lysates using anti-human MMP1 antibody without IPTG induction (lane 1) and with 1 mM IPTG induction (lane 2). (4C) SDS PAGE of trypsin-treated supernatant of cell lysates showing activation of MMP1. MW markers (lane 1), 54 kD pro-MMP1 expression in the untreated supernatant (lane 2, indicated by the black arrow), trypsin-activated MMP1 (43 kD band indicated by the arrow). (4D) SDS PAGE of the pellet after dissolving in 8 M urea. MW markers (lane 1), MMP1 in the pellet (lane 2). The arrow indicates truncated 28 kD MMP1 that forms inclusion body and precipitates. (4E) Western blot of the pellet after centrifugation of cell lysates.

FIGS. 5A-C illustrate the purification and identification of MMP1. (5A) SDS PAGE gel of purified active MMP1 after trypsin activation and centrifugation using Amicon filter. Molecular weight markers (lane 1) and purified active MMP1 (lane 2). (5B) Western blot of active MMP1. (5C) MALDI-TOF mass spectrum of active MMP1. (Inset) Fragmentation of MMP1 during the mass spectrometry.

FIGS. 6A-C illustrate collagen degradation activities of both active and catalytically inactive mutant MMP1. (6A) Coomasssie-stained SDS PAGE gel to show concentration dependent activity. Molecular weight (MW) markers (lane 1), FITC-labeled bovine type-1 collagen (lane 2), Lanes 3, 4, 5 are 100 μg collagen+MMP1 at 25 μg, 50 μg, and 100 μg respectively. Black arrows (middle of gel) point to ¾ TC^{A} fragments, whereas arrows at the lower portion of the gel point to ¼ TC^{B} fragments. (6B) Fluorescent image of the same gel in FIG. 6a. MW markers (lane 1), FITC-labeled bovine type-1 collagen (lane 2), lanes 3, 4, 5 are 100 μg collagen+MMP1 at 25 μg, 50 μg, and 100 μg respectively. (6C) Gelatin zymogram of MMP1 activity. MMP1 4 μg (lane 1), MMP1 6 μg (lane 2), and MMP1 8 μg (lane 3). Zymogram shows the complex and active MMP1 at 28 kD instead of 43 kD because of thermal fragmentation.

FIGS. 7A-B illustrate the specific activity of MMP1 using fluorogenic peptide substrate. (7A) Relative fluorescence unit (RFU) of degraded substrate by 0.05 μg MMP1 as a function of time (circle) and the best fit (blue line) to y=a−b exp(−kt), a general solution to the Michaelis-Menten equation; the fit parameters are a=25.73±0.16, b=23.46±0.89, and the reaction rate, k=0.26±0.02 RFU/min. The specific activity is k/0.05=5.2 RFU/min/μg. (7B) RFU of the substrate at different concentrations (circle) and the best fit (black line) to y=mx+c; the fit parameters are m=0.50±0.03 and c=0.24±1.01. The calibration curve translates 5.2 RFU into ˜999 pmol of substrate and the specific activity to ˜999 pmol/min/μg. The error bars in data points represent the standard deviations of 6 repeats, whereas the error bars in the parameters are the standard deviations of fits.

FIG. 8 illustrates a fluorescent image of SDS PAGE gel to compare active MMP1 with catalytically inactive mutant MMP1 and trypsin. MW markers (lane 1), FITC-labeled bovine type-1 collagen (lane 2), collagen+trypsin (lane 3), collagen+trypsin+trypsin inhibitor (lane 4), collagen+active MMP1 (lane 5), collagen+active MMP1+trypsin inhibitor (lane 6), collagen+mutant MMP1 (lane 7), collagen+mutant MMP1+trypsin inhibitor (lane 8), and collagen+commercial MMP1 (lane 9).

FIGS. 9A-B illustrate broad-spectrum protease activity of MMP1 on native E. coli proteins. (9A) Effect of MMP1 on native proteins produced by E. coli strain without MMP1 plasmid. Molecular weight (MW) markers (lane 1), supernatant of lysate (lane 2), supernatant treated with MMP1 only (lane 3), supernatant treated with trypsin only (lane 4), and supernatant treated with both MMP1 and trypsin (lane 5). (9B) Effect of MMP1 on proteins produced by E. coli strain with MMP1 plasmid. Molecular weight markers (lane 1), supernatant of lysate (lane 2), supernatant treated with MMP1 only (lane 3), treated with trypsin only (lane 4), and treated with both MMP1 and trypsin (lane 5). Black arrows indicate the presence of MMP1 in recombinant E. coli supernatant.

FIGS. 10A-B illustrate the protease activity of MMP1 purified using sonication to lyse Rosetta (DE3) pLysS cells. (10A) Empty Rosetta (DE3) pLysS without plasmid. (10B) Rosetta (DE3) pLysS cells with the MMP1 gene. For both Figs. S10a and S10b: molecular weight markers (lane 1), untreated supernatant (lane 2), MMP1-treated supernatant (lane 3), trypsin-treated supernatant (lane 4), and trypsin- and MMP1-treated (lane 5).

FIG. 11 illustrates an SDS PAGE gel showing the effect of active MMP1, trypsin, mutant MMP1, and commercial MMP1 on whole cell lysate of E. coli strains with MMP1 plasmid. MW markers (lane 1), Cell lysate (lane 2), cell lysate+trypsin (lane 3), cell lysate+trypsin+trypsin inhibitor (lane 4), cell lysate+active MMP1 (lane 5), cell lysate+active MMP1+trypsin inhibitor (lane 6), cell lysate+mutant MMP1 (lane 7), cell lysate+mutant MMP1+trypsin inhibitor (lane 8), and cell lysate+commercial MMP1 (lane 9).

FIG. 12 illustrates a general method of the present invention of purifying MMP1.

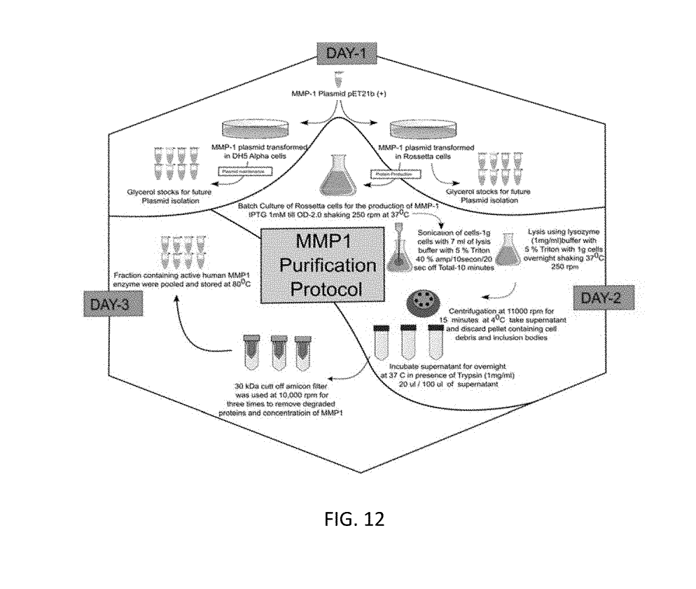

FIG. 13 shows the sequence of cDNA encoding MMP9 (SEQ ID NO:4). Rare codons (relative to E. coli) are bolded and underlined.

### DETAILED DESCRIPTION OF THE INVENTION

As has been discussed, current methods for purifying certain proteins, such as MMP proteins, are expensive and/or result in low yields of active protein. The present invention provides inexpensive, high-throughput methods for purifying active proteins of interest from recombinant, including bacterial cells such as Escherichia coli (E. coli). A method of the invention can generally be practiced by increasing the initial expression level of a protein of interest (POI) in a recombinant cell, and then purifying the POI from the cell using a non-ionic detergent and filtration techniques. In certain aspects of the invention, proteases may be utilized concurrently during purification of the POI from the cell. In certain aspects of the invention, the protein of interest is a matrix metalloproteinase protein (MMP).

The terminology used herein is for the purpose of describing particular embodiments only, and is not intended to be limiting, since the scope of this disclosure will be limited only by the claims.

As used herein and in the appended claims, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Unless defined otherwise, all technical and scientific terms used herein have the same meaning as commonly understood by one of ordinary skill in the art to which this invention belongs. Although any methods and materials similar or equivalent to those described herein can also be used in the practice or testing of this disclosure, the preferred methods and materials are now described.

One aspect of the invention is a method of purifying a protein of interest (POI), the method comprising

a. culturing a recombinant cell comprising an expression vector that comprises a nucleic acid sequence encoding the POI, under conditions suitable for production of the POI;

b. lysing the cells in the presence of a non-ionic detergent to produce a lysate;

c. contacting the soluble portion of the lysate with a protease to which the POI is resistant; and

d. contacting the soluble portion of the lysate with a filter to produce a retentate, and a filtrate containing the POI, thereby purifying the POI.

Any protein of interest (POI) can be purified using the disclosed method, as long as the protein of interest is resistant to at least one protease. The protein of interest may be naturally resistant to at least one protease. Alternatively, the protein of interest may be made resistant to one or more proteases by altering the sequence of the protein. In certain aspects, in modifying a protein of interest so that it is resistant to one or more protease, the modification should not significantly affect the activity (e.g., catalytic, enzymatic, etc.) of the protein. Thus, the modification should not alter the activity by more than about 10%, about 20%, or about 30%. In certain aspects, modification of the sequence of the protein of interest may produce an inactive protein.

One aspect of the invention is a method of purifying a matrix metalloproteinase protein (MMP), the method comprising:

a. culturing a recombinant cell comprising an expression vector that comprises a nucleic acid sequence encoding the MMP protein, under conditions suitable for production of the MMP protein;

b. lysing the cells in the presence of a non-ionic detergent to produce a lysate;

c. contacting the soluble portion of the lysate with a serine protease; and

d. contacting the soluble portion of the lysate with a filter to produce a retentate and a MMP containing filtrate, thereby purifying the MMP protein.

In such methods, any cell can be used as long as the cell can effect expression of the protein of interest (e.g., an MMP protein) from the expression vector. Examples of cells useful for practicing the invention include, but are not limited to, fibroblasts, mouse myeloma cells, Chinese hamster ovary (CHO) cells, yeast cells, and bacterial cells. In certain aspects, bacterial cells are preferred cells to use. A particularly useful bacterial cell is an E. coli cell. It will be understood by those skilled in the art that cells used in practicing the invention should contain the necessary elements (e.g., enzymes, promoters, etc.) to effect transcription of the nucleic acid sequence encoding the protein of interest. Such cells are known in the art, examples of which include, but are not limited to, BL21 cells, HMS174 cells, Rosetta™ and T7 Express cells.

Any expression vector may be used in practicing the disclosed methods, as long as the expression vector is capable of expressing the POI (e.g., MMP) in the chosen cell. Examples of suitable expression vectors include, but are not limited to, viral vectors, phages, cosmids, and plasmids. It will be understood by those skilled in the art that the vector should be appropriately matched to the chosen cell. For example, if the cell used in the method is a bacterial cell, a suitable vector would be a bacterial expression vector, such as a plasmid. Such plasmids are known in the art and are commercially available. One example of such an expression vector is the pET expression system, available from Millipore Sigma. In one aspect of the invention, the expression vector may be a pET expression vector, such as, pET21b.

Any matrix metalloproteinase protein may be purified using the disclosed methods. Examples of such proteins include, but are not limited to MMP1, MMP2, MMP3, MMP7, MMP8, MMP9, and MMP10. In one aspect, MMP1 may be purified. In one aspect, MMP9 may be purified.

Expression vectors used in the disclosed method may comprise a nucleic acid sequence encoding an MMP protein. In certain aspects, the MMP protein may comprise, or consist of, an amino acid sequence at least 80% identical, at least 85% identical, at last 90% identical, at least 95% identical, at least 96% identical, at least 97% identical, at least 98% identical, or at least 99% identical to SEQ ID NO:3, SEQ ID NO:6 or SEQ ID NO:8. In one aspect, the MMP protein may comprise SEQ ID NO:3. In one aspect, the MMP protein may comprise SEQ ID NO:6. In one aspect, the MMP protein may comprise SEQ ID NO:8.

In certain aspects of the invention, the nucleic acid sequence encoding the protein of interest (e.g., the MMP protein) may be codon-optimized. It is known in the art that at least some amino acids are encoded by more than one codon. Consequently, cells contain several species of tRNAs that recognize the same amino acid and have similar, but not identical, anti-codon sequences. In different organisms, the populations of these different tRNA species vary, with some species being more abundant in one type of cell than in another type of cell. If a cell contains a limiting number of tRNA species for a particular amino acid, the expression of any nucleic acid sequence containing a codon recognized by the limited tRNA species may be reduced in that cell. Codons recognized by limited, or absent, tRNA species are referred to as rare codons. As used herein, codon optimization, codon optimized, and the like, refer to a process in which one or more rare codons are altered (mutated) (e.g., substitution mutation) so that the resulting codon(s) is/are not rare. That is, the resulting codon(s) is/are recognized by a tRNA species that is more, and preferably most, abundant in the cell used for expression of the nucleic acid sequence, than is the tRNA species that recognizes the rare codon(s). Codon optimization does not change the amino acid sequence encoded by the nucleic acid sequence. Thus, it should be understood that the process of codon optimization introduces silent mutations into the codon(s) being altered. Methods of codon optimization are known to those skilled in the art, and are also disclosed in US20070292918A1, which is incorporated herein by reference, in its entirety.

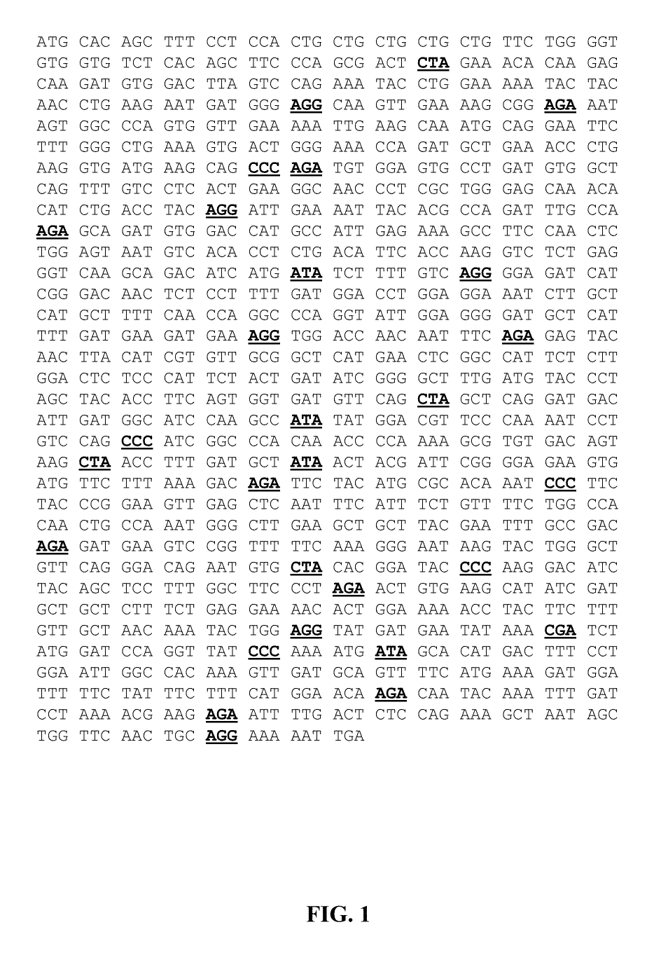

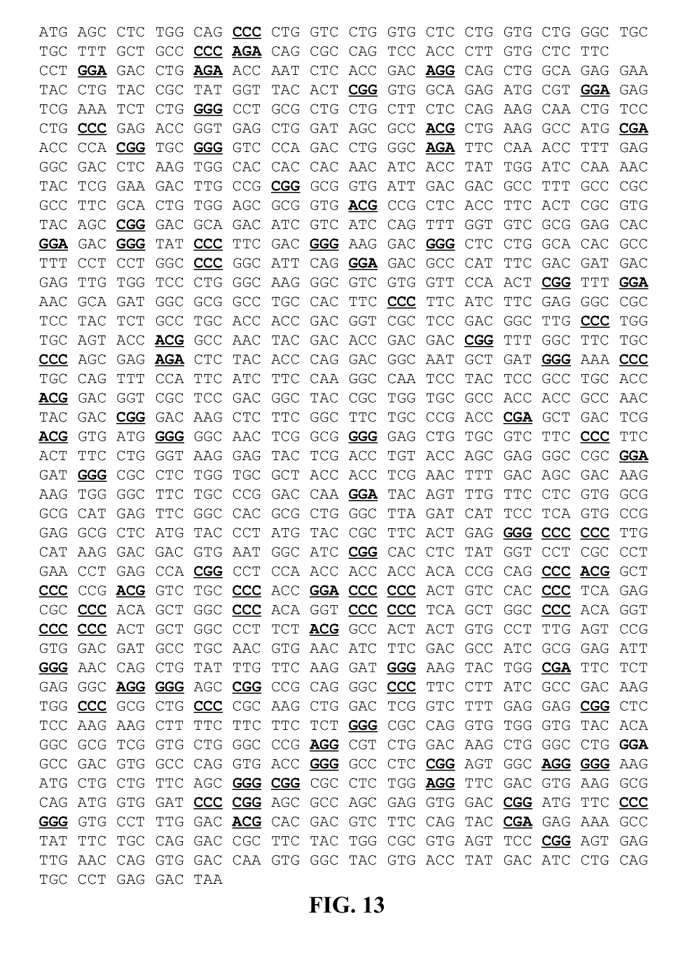

In certain aspects, the expression vector used in the claimed method comprises a nucleic acid sequence at least at least about 70% identical, at least about 80% identical, at least about 85% identical, at last about 90% identical, at least about 95% identical, at least about 96% identical, at least about 97% identical, at least about 98% identical, at least about 99% identical, or at least 100% identical to SEQ ID NO:2, SEQ ID NO:5, or SEQ ID NO:7 wherein the nucleic acid sequence has been codon optimized. In certain aspects, the nucleic acid sequence may be codon optimized for expression in a mammalian cell. In certain aspects, the nucleic acid sequence may be codon optimized for expression in a yeast cell. In certain aspects, the nucleic acid sequence may be codon optimized for expression in a bacterial cell. In certain aspects, the nucleic acid sequence may be codon optimized for expression in E. coli cells. The nucleic acid sequence may be optimized at one or more codons indicated (bold/underline) in FIG. 1 or FIG. 13.

In certain aspects of the invention, the expression vector comprises a nucleic acid sequence at least at least about 70% identical, at least about 80% identical, at least about 85% identical, at last about 90% identical, at least about 95% identical, at least about 96% identical, at least about 97% identical, at least about 98% identical, at least about 99% identical to SEQ ID NO:1 or SEQ ID NO:4, wherein the difference between the nucleic acid sequence and SEQ ID NO:1 or SEQ ID NO:4, is due, at least in part, to silent mutations in one or more rare codons of the nucleic acid sequence. The nucleic acid sequence may be at least at least about 70% identical, at least about 80% identical, at least about 85% identical, at last about 90% identical, at least about 95% identical, at least about 96% identical, at least about 97% identical, at least about 98% identical, at least about 99% identical to SEQ ID NO:1, wherein the difference between the nucleic acid sequence and SEQ ID NO:1 is due, at least in part, to silent mutations in one or more codons of the nucleic acid sequence that correspond to one or more rare codons indicated bold and/or underlined) in FIG. 1. The nucleic acid sequence may be at least at least about 70% identical, at least about 80% identical, at least about 85% identical, at last about 90% identical, at least about 95% identical, at least about 96% identical, at least about 97% identical, at least about 98% identical, at least about 99% identical to SEQ ID NO:1, wherein the difference between the nucleic acid sequence and SEQ ID NO:1 is due, at least in part, to silent mutations in one or more codons of the nucleic acid sequence that correspond to one or more rare codons in SEQ ID NO:1 selected from the group consisting of codon 24, codon 49, codon 55, codon 90, codon 91, codon 161, codon 165, codon 202, codon 208, codon 248, codon 259, codon 269, codon 282, codon 287, codon 300, codon 307, codon 337, codon 357, codon 361, codon 372, codon 399, codon 405, codon 412, codon 415, codon 443, codon 453, and codon 467. In certain aspects, the nucleic acid sequence may lack mutations in codons encoding amino acids in the active site, thereby encoding an active MMP.

In certain aspects of the invention, the expression vector used in the claimed method comprises a nucleic acid sequence at least at least about 70% identical, at least about 80% identical, at least about 85% identical, at last about 90% identical, at least about 95% identical, at least about 96% identical, at least about 97% identical, at least about 98% identical, at least about 99% identical to SEQ ID NO:4, wherein the difference between the nucleic acid sequence and SEQ ID NO:4 is due, at least in part, to silent mutations in one or more codons that correspond to one or more rare codons indicated (bold/underlined) in FIG. 13. The nucleic acid sequence may be at least at least about 70% identical, at least about 80% identical, at least about 85% identical, at last about 90% identical, at least about 95% identical, at least about 96% identical, at least about 97% identical, at least about 98% identical, at least about 99% identical to SEQ ID NO:4, wherein the difference between the nucleic acid sequence and SEQ ID NO:4 is due, at least in part, to silent mutations in one or more codons of the nucleic acid sequence that correspond to one or more rare codons in SEQ ID NO:4 selected from the group consisting of codon 6, codon 21, codon 22, codon 33, codon 36, codon 42, codon 56, codon 62, codon 68, codon 81, codon 90, codon 95, codon 98, codon 100, codon 106, codon 134, codon 152, codon 162, codon 176, codon 178, codon 180, codon 183, codon 186, codon 196, codon 200, codon 221, codon 223, codon 233, codon 254, codon 259, codon 267, codon 275, codon 285, codon 287, codon 304, codon 322, codon 332, codon 336, codon 339, codon 344, codon 350, codon 367, codon 369, codon 392, codon 428, codon 429, codon 430, codon 440, codon 452, codon 461, codon 462, codon 464, codon 466, codon 469, codon 471, codon 472, codon 473, codon 477, codon 481, codon 485, codon 488, codon 489, codon 493, codon 496, codon 497, codon 503, codon 528, codon 537, codon 541, codon 546, codon 547, codon 549, codon 553, codon 561, codon 564, codon 574, codon 584, codon 599, codon 607, codon 615, codon 618, codon 621, codon 622, codon 629, codon 630, codon 634, codon 644, codon 645, codon 652, codon 655, codon 656, codon 661, codon 668, and codon 685. In certain aspects, the nucleic acid sequence may lack mutations in codons encoding amino acids in the active site, thereby encoding an active MMP.

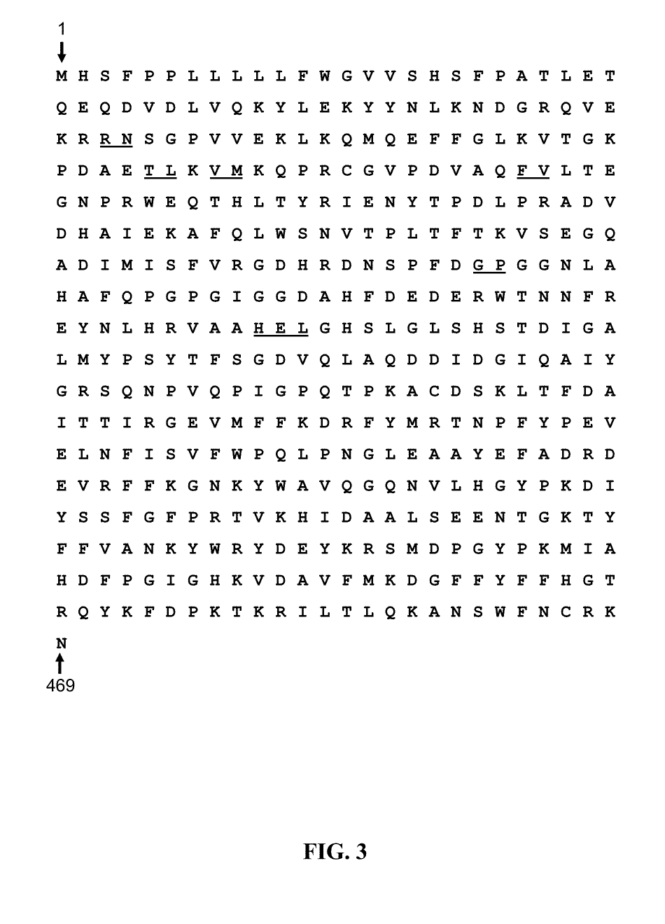

In certain aspects, the MMP encoded by the nucleic acid sequence may contain a mutation in the active site, resulting in an inactive protein. The active site of MMP1 consists of the amino acids HEL (see FIG. 3) and is encoded by codons 218, 219, and 220 of SEQ ID NO:1. The active site of MMP9 consists of the amino acids HQF (see FIG. 3) and is encoded by codons 401, 402, and 403 of SEQ ID NO:7. Thus, in certain aspects, the nucleic acid sequence encoding the MMP may comprise a mutation in a codon encoding an amino acid residue in the active site of the MMP, such that one or more amino acid residues in the active site are deleted or substituted with a different amino acid residue, resulting in an inactive MMP. The nucleic acid sequence may comprise a mutation in a codon corresponding to any one of codons 218-220 of SEQID NO:1 or codons 401-402 of SEQ ID NO:7. The nucleic acid sequence may be at least at least about 70% identical, at least about 80% identical, at least about 85% identical, at last about 90% identical, at least about 95% identical, at least about 96% identical, at least about 97% identical, at least about 98% identical, at least about 99% identical to SEQ ID NO:1, wherein the nucleic acid sequence comprises a mutation in a codon corresponding to any one of codons 218-220 of SEQID NO:1. The nucleic acid sequence may be at least at least about 70% identical, at least about 80% identical, at least about 85% identical, at last about 90% identical, at least about 95% identical, at least about 96% identical, at least about 97% identical, at least about 98% identical, at least about 99% identical to SEQ ID NO:4, wherein the nucleic acid sequence comprises a mutation in a codon corresponding to any one of codons 401-402 of SEQID NO:4.

In certain aspects of the invention, the expression vector used in the claimed method comprises a nucleic acid sequence at least at least about 70% identical, at least about 80% identical, at least about 85% identical, at last about 90% identical, at least about 95% identical, at least about 96% identical, at least about 97% identical, at least about 98% identical, at least about 99% identical, or at least 100% identical to SEQ ID NO:7, wherein the nucleic acid sequence comprises a silent mutation in one or more codons that correspond to one or more rare codons indicated (bold and/or underlined) in FIG. 13, and wherein the nucleic acid sequence comprises a mutation in any one of codons 401-403, such that the encoded MMP is inactive. The nucleic acid sequence may be at least at least about 70% identical, at least about 80% identical, at least about 85% identical, at last about 90% identical, at least about 95% identical, at least about 96% identical, at least about 97% identical, at least about 98% identical, at least about 99% identical, or at least 100% identical to SEQ ID NO:7, wherein the nucleic acid sequence comprises a silent mutation in one or more codons that correspond to one or more rare codons in SEQ ID NO:4 selected from the group consisting of codon 6, codon 21, codon 22, codon 33, codon 36, codon 42, codon 56, codon 62, codon 68, codon 81, codon 90, codon 95, codon 98, codon 100, codon 106, codon 134, codon 152, codon 162, codon 176, codon 178, codon 180, codon 183, codon 186, codon 196, codon 200, codon 221, codon 223, codon 233, codon 254, codon 259, codon 267, codon 275, codon 285, codon 287, codon 304, codon 322, codon 332, codon 336, codon 339, codon 344, codon 350, codon 367, codon 369, codon 392, codon 428, codon 429, codon 430, codon 440, codon 452, codon 461, codon 462, codon 464, codon 466, codon 469, codon 471, codon 472, codon 473, codon 477, codon 481, codon 485, codon 488, codon 489, codon 493, codon 496, codon 497, codon 503, codon 528, codon 537, codon 541, codon 546, codon 547, codon 549, codon 553, codon 561, codon 564, codon 574, codon 584, codon 599, codon 607, codon 615, codon 618, codon 621, codon 622, codon 629, codon 630, codon 634, codon 644, codon 645, codon 652, codon 655, codon 656, codon 661, codon 668, and codon 685, and wherein the nucleic acid sequence comprises a mutation in any one of codons 401-403, such that the encoded MMP is inactive.

It should be understood that mutating the MMP encoding nucleic acid sequence to optimize the codon usage results in an optimized nucleic acid sequence, the expression of which results in an increase in the amount of MMP protein produced, relative to the amount of MMP protein produced from a non-optimized nucleic acid sequence encoding MMP protein. As used herein, an increased amount of MMP protein means an increase of at least 10%, at least 20%, at least 30%, at least 40%, at least 50%, at least 60%, at least 70%, at least 80%, at least 90%, at least 100%, or at least 200%. In one aspect of the invention, the expression vector used in the claimed method comprises a nucleic acid sequence comprising, or consisting, of SEQ ID NO:2, SEQ ID NO:4 or SEQ ID NO:7.

Examples of sequences useful for practicing methods of the disclosure are shown below in Table 1.

TABLE 1

| SEQ | | |
|---|---|---|
| ID | | |
| NO | Molecule | Description |
| 1 | DNA | cDNA sequence of MMP1 |
| 2 | DNA | Optimized coding sequence for MMP1 |
| 3 | Protein | Amino acid sequence encoded by SEQ ID Nos 1 and 2 |
| 4 | DNA | cDNA sequence of MMP9 |
| 5 | DNA | Optimized coding sequence for MMP9 |
| 6 | Protein | Amino acid sequence encoded by SEQ ID Nos 4 and 5 |
| 7 | DNA | Optimized coding sequence for MMP9 Active site |
| | | (E402Q) mutant |
| 8 | Protein | Amino acid sequence for MMP9 Active Site (E402Q) |
| | | mutant |

In certain aspect, the nucleic acid sequence encoding the protein of interest (e.g., the MMP protein) is functionally linked to a promoter in the expression vector. As used herein, functionally linked means that proteins encoded by the linked nucleic acid molecules can be expressed when the linked promoter is activated. Promoters useful for practicing the present invention are known to those skilled in the art. Examples of useful promoters include, but are not limited to a T7 promoter, a trp promoter, a lac promoter, an SP6 promoter, and a CMV promoter.

One aspect of the invention is a recombinant cell comprising an optimized MMP encoding nucleic acid molecule of the present invention. One aspect of the invention is a recombinant virus comprising an optimized MMP encoding nucleic acid molecule of the present invention.

To prevent the aggregation of the protein of interest (e.g., the MMP protein) in inclusion bodies and drive the dynamic equilibrium of a supernatant toward the soluble form of the protein of interest, in methods of the invention, cells comprising the expression vector are lysed in the presence of a non-ionic detergent. Any nonionic detergent may be used as long as it is capable of lysing the cell without inactivating the expressed protein of interest (e.g., MMP1). Such non-ionic detergent may be a polyoxyethylene, a polysorbate or a non-ionic surfactant. In certain aspects, the non-ionic detergent may be selected from the group consisting of Tween, Triton®, and Nonidet-P40. In certain aspects, the non-ionic detergent may be Triton® X-100.

In such methods, the cells are lysed to release the expressed protein of interest. Any method of lysis may be used, as long as the released protein of interest retains the desired activity (e.g., an MMP protein should retain metalloproteinase activity). For example, the step of lysing the cells may comprise sonification, French press and/or chemical lysis (e.g., lysozyme).

The protease contacted with the soluble portion of the lysate should be a protease to which the protein of interest is resistant. That is, the protease is unable to cut the protein of interest. The protease may be a serine protease. The serine protease may be trypsin, chymotrypsin, trypsinogen, or pepsinogen. The protease may be contacted with the soluble portion of the lysate by adding protease from an external source (e.g., protease obtained from a commercial manufacture). Alternatively, a second expression vector encoding a protease may be introduced into the cells used for expression of the protein of interest. Induction of the encoded proteases would result in production of the proteases within the cell. Following lysis, the protease may be activated, for example using MMP, which will result in degradation of the cellular proteins in the soluble portion of the lysate.

Lysis of the cells produces a lysate, containing the soluble protein, and an insoluble fraction. In certain aspects of the invention, the lysate may be separated from the insoluble portion using techniques such as, filtration or centrifugation. In certain aspects, filtration, or centrifugation, may be performed at a temperature less than room temperature (e.g., less than 23° C.) In certain aspects, filtration, or centrifugation, may be performed at a temperature of less than 12° C.

In certain aspects, once the lysate is separate from the insoluble fraction, it may be further filtered. In certain aspects, the filter has a molecular weight cutoff of 30 kDA.

One aspect of the invention is a nucleic acid molecule comprising a nucleic acid sequence encoding an active, or inactive, MMP, wherein the nucleic acid sequence has been codon optimized. The MMP may be MMP1, MMP2, MMP3, MMP7, MMP8, MMP9, or MMP10. The nucleic acid sequence may comprise a mutation in a codon encoding an amino acid residue in the active site of the MMP. The nucleic acid sequence may be at least at least about 70% identical, at least about 80% identical, at least about 85% identical, at last about 90% identical, at least about 95% identical, at least about 96% identical, at least about 97% identical, at least about 98% identical, at least about 99% identical to SEQ ID NO:1, wherein the difference between the nucleic acid sequence and SEQ ID NO:1 is due, at least in part, to a silent mutation in one or more codons of the nucleic acid sequence that correspond to one or more rare codons in SEQ ID NO:1 selected from the group consisting of codon 24, codon 49, codon 55, codon 90, codon 91, codon 161, codon 165, codon 202, codon 208, codon 248, codon 259, codon 269, codon 282, codon 287, codon 300, codon 307, codon 337, codon 357, codon 361, codon 372, codon 399, codon 405, codon 412, codon 415, codon 443, codon 453, and codon 467. The nucleic acid sequence may be at least at least about 70% identical, at least about 80% identical, at least about 85% identical, at last about 90% identical, at least about 95% identical, at least about 96% identical, at least about 97% identical, at least about 98% identical, at least about 99% identical to SEQ ID NO:4, wherein the difference between the nucleic acid sequence and SEQ ID NO:4 is due, at least in part, to a silent mutation in one or more codons of the nucleic acid sequence that correspond to one or more rare codons in SEQ ID NO:4 selected from the group consisting of codon 6, codon 21, codon 22, codon 33, codon 36, codon 42, codon 56, codon 62, codon 68, codon 81, codon 90, codon 95, codon 98, codon 100, codon 106, codon 134, codon 152, codon 162, codon 176, codon 178, codon 180, codon 183, codon 186, codon 196, codon 200, codon 221, codon 223, codon 233, codon 254, codon 259, codon 267, codon 275, codon 285, codon 287, codon 304, codon 322, codon 332, codon 336, codon 339, codon 344, codon 350, codon 367, codon 369, codon 392, codon 428, codon 429, codon 430, codon 440, codon 452, codon 461, codon 462, codon 464, codon 466, codon 469, codon 471, codon 472, codon 473, codon 477, codon 481, codon 485, codon 488, codon 489, codon 493, codon 496, codon 497, codon 503, codon 528, codon 537, codon 541, codon 546, codon 547, codon 549, codon 553, codon 561, codon 564, codon 574, codon 584, codon 599, codon 607, codon 615, codon 618, codon 621, codon 622, codon 629, codon 630, codon 634, codon 644, codon 645, codon 652, codon 655, codon 656, codon 661, codon 668, and codon 685. The nucleic acid sequence at least at least about 70% identical, at least about 80% identical, at least about 85% identical, at last about 90% identical, at least about 95% identical, at least about 96% identical, at least about 97% identical, at least about 98% identical, at least about 99% identical, or at least 100% identical to SEQ ID NO:2, SEQ ID NO:5, or SEQ ID NO:7. In certain aspects, the nucleic acid sequence may lack mutations in codons encoding amino acids in the active site, thereby encoding an active MMP. In certain aspects, the nucleic acid sequence may comprise a mutation in codons encoding amino acids in the active site, thereby encoding an inactive MMP.

### EXAMPLES

The following example is put forth so as to provide those of ordinary skill in the art with a complete disclosure and description of how to make and use the embodiments. This examples is not intended to limit the scope of what the inventors regard as their invention nor are they intended to represent that the experiments below are all of the, or the only, experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, and temperature is in degrees Celsius. Standard abbreviations are used. All values listed in the Examples are approximate.

### Methods

Optimization of MMP1 cDNA for Expression in E. coli

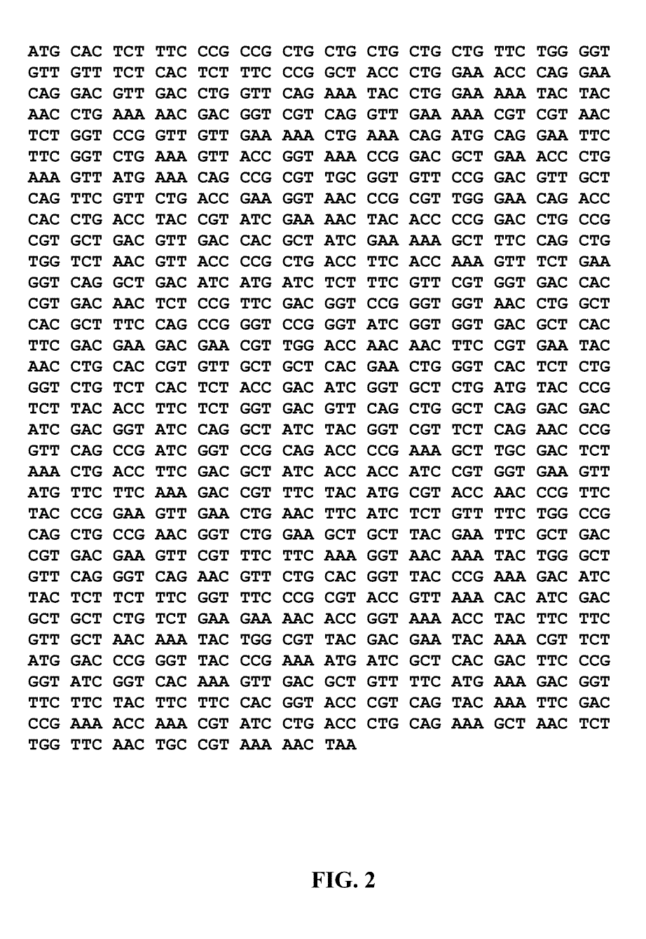

Rare codons in the cDNA sequence of MMP1 were identified using the Rare Codon Calculator (RaCC) tool of the NIH MBI Laboratory for Structural Genomics and Proteomics (http://nihserver.mbi.ucla.edu/RACC). The codons for arginine (AGG, AGA, and CGA), leucine (CTA), isoleucine (ATA), and proline (CCC) were identified as rare in the cDNA. The locations of these codons in the MMP-1 cDNA is shown in FIG. 1. To remove rare codons, the DNA sequence of MMP1 was optimized for expression in E. coli using the online Java codon adaptation tool (JCat) (http://www.JCat.de) by selecting E. coli K12 as the organism. Analysis using JCat showed that the codon adaptation index (CAI) and the GC content of the optimized MMP1 gene is 0.2 and 45.2, which are not optimal. Hence, the codon adaptation index value was optimized to 1.0, which is ideal for proteins expressed in E. coli. After optimization, the sequence was checked again, which showed that all rare codons were completely removed from the MMP1 gene sequence (FIG. 2). The CAI value and the GC content of the optimized MMP1 gene were 1.0 and 51.4 respectively. The amino acid sequence before and after optimization remained unchanged.

### Construction and Transformation of Expression Plasmid

The optimized MMP1 sequence was synthesized by General Biosystems and inserted into the pET-21b (+) vector between NdeI (N-terminal) and HindIII (C-terminal) restriction sites. The plasmid was transformed into Rosetta (DE3) pLysS Competent Cells competent cells E. coli (Novagen, Cat#70956). Bacteria were thawed for 10 min on ice and 5 μl of plasmid (100 ng/μ1) added to 50 μl cells. Bacteria were incubated on ice for 30 min followed by heat shock at 42° C. in an ultrasonic water bath (Panasonic, CPX 2800H) for 45 s followed by a second incubation on ice for 5 min. 900 μl of super optimal broth with catabolite repression (SOC) media pre-warmed at 37° C. (NEB, Cat# B9020S) was added to the reaction mixture and placed in an incubator at 37° C. (Thermo Scientific MaxQ 600, Model#4353) for 1 hr at 250 rpm. ImMedia Amp Blue plates (Thermo Fisher Scientific, Cat# Q60120) were prepared according to manufacturer's instructions. Heated ImMedia was cooled to 37° C. and 20 ml plated on each sterile disposable petri plate. 500 μl of the transformation reaction mixture was spread using a sterilized disposable L-shaped plastic rod on the ImMedia plates containing X-gal and ampicillin. The plates were incubated at 37° C. for 15 hr to allow colony growth. Transformation was successful in both DH5alpha and Rosetta PLysS. After transformation glycerol stocks of E. coli strains were stored at −80° C.

Culture of Rosetta (DE3) pLysS Cells

35 ml of sterile LB media in a sterile falcon tube was inoculated with a single colony from the Rosetta (DE3) pLysS plate in a sterile environment. Chloramphenicol (Sigma, Cat# C0378) and ampicillin (Sigma, Cat# A9518) were added to a final concentrations of 34 μg/μl and 100 μg/μl respectively. Following inoculation, the culture was incubated at 250 rpm to 15 hr at 37° C. (OD₆₀₀=1.5-2.0). 10 ml of the culture was added to 400 ml of sterile LB in a 1 liter conical flat bottom glass flasks and cells were grown to OD₆₀₀=0.1 (pH 7.0) at 37° C. at 250 rpm in the presence of chloramphenicol and ampicillin added to a final concentration of 34 μg/μl and 100 μg/μl respectively. After the culture reached OD₆₀₀=0.1, the cells were induced with 1 mM Isopropyl β-D-1-thiogalactopyranoside (IPTG). The cells without IPTG were processed similarly and served as the uninduced control. The cells were induced for 5 hr until the OD₆₀₀ reached 1.8 (pH 6.7) at 37° C. 200 μM ZnCl₂ and 400 μM CaCl₂) were added to the flask at the time of induction. The optical density was checked by a cell density meter (WPA Biowave, Model# C0800). The cells were harvested and centrifuged in 50 ml centrifuge tubes at 10000 rpm for 10 minutes using a fixed angle centrifuge (Sorvall Lynx 4000 centrifuge with F12-6X500 rotor, Cat#75006580).

### Cell Lysis and SDS PAGE

Two methods of lysis were used. The first method used lysozyme. 1 g of the centrifuged cells was reconstituted in 7 ml of lysis buffer (pH 9) containing 50 mM Tris base (Sigma, Cat# T4661), 100 mM NaCl, 200 μM ZnCl₂, 400 μM CaCl₂), 5% Triton® X-100, and 1 mg/ml lysozyme. The reconstituted cells were incubated at 37° C. at 250 rpm. Only freshly prepared Triton® X-100 solution was used in all the buffers. The second method lysed the cells via sonication. The cells were disrupted using a sonicator (Branson Digital Sonifier, Model# BBT16031593A) at 40% amplitude for 10 min with a sequence of 10 s pulse ON and 20 s pulse OFF. The sample was immersed in an ice-water bath during the sonication to reduce the heating effect. For both methods, the induced cell lysate with 1 mM IPTG appeared creamy white, while the uninduced lysate without 1 mM IPTG appeared brownish. The lysate was centrifuged at 11000 rpm for 15 min. Both the precipitated pellet and supernatant were analyzed using SDS PAGE. The samples were mixed with 2× Laemmli sample buffer (Biorad, Cat#610737) in 3:1 ratio. To reduce disulphide bonds and complex formation, β-mercaptoethanol (BME) and urea were added to obtain the final concentrations of 10% v/v and 8 M respectively. The samples were not heated since heating leads to MMP1 breaks. The samples were loaded onto a 12% resolving polyacrylamide gel and subjected to electrophoresis in Tris-glycine SDS buffer for 30 min at 50 V followed by 1 hr at 120 V. 15 μl of either the Precision Plus protein kaleidoscope pre-stained protein standards (Biorad, Cat#1610375) or the Precision Plus unstained protein standards (Biorad, Cat#1610363) was used as molecular weight markers. The gels were stained with Coomassie Brilliant Blue R-250 (Biorad, Cat#161-0436) for 15 hr on an orbital shaker at room temperature. After staining, SDS PAGE gel was paced on A4 size ultra-thin portable LED light box tracer and imaged with a camera (Canon Coolpix, SX100). All images were captured using the default image setting of the camera. Images were copied and pasted in Microsoft PowerPoint. Gel image brightness was set at 20% and image contrast was set at 40% by double clicking on the image and then clicking on the correction tab.

### Activation and Purification

Both trypsin and chymotrypsin were used to activate MMP1. The supernatant was treated with either trypsin or chymotrypsin at the final concentration of 0.1 mg/ml. The activation reaction occurred in a flat bottom conical flask for 15 hr at 37° C. at 250 rpm. For activation and purification with previously purified MMP1, the final concentration of 0.01 μg/μl of MMP1 was used. The digested proteins, lysozyme, and trypsin were removed by centrifugation using 30 kD cut-off filters three times and once with a 100 kD cut-off filter at 8000 rpm at 4° C. After each centrifugation, dilution buffer without Triton® was added to achieve the initial volume because Triton® X-100 did not pass through the 30 kD filter. The resultant MMP1 was quantified using Bradford assay and analyzed by SDS PAGE.

### Identification of MMP1 Using Western Blotting

The protein was transferred to a PVDF membrane at 100 V for 2 hr at 4° C. in the transfer buffer containing 25 mM Tris base, 190 mM glycine, 20% methanol, and 0.1% SDS. The Western blot assembly was placed in a large ice bath to reduce the temperature, and thus the fragmentation of MMP1, during transfer. After transferring the protein, the blot was rinsed in TBS wash buffer without Tween 20. Once washed, the membrane was incubated in a blocking buffer (skim milk) for 2 hr. It should be noted that the presence of Tween 20 in the transfer buffer caused precipitation of the protein. The PVDF membrane was rinsed a second time with the wash buffer and incubated overnight at 4° C. on an orbital shaker with the primary anti-human MMP1 antibody (BosterBio, anti-MMP1 polyclonal IgG antibody raised in rabbit, Cat# PB9725) diluted in the blocking buffer at the final concentration of 1 ug/ml. The blot was rinsed with the wash buffer 3 more times for 1 min each. After washing, the membrane was incubated with the HRP-conjugated anti-rabbit IgG antibody (BosterBio, Cat# BA1070-0.5) for 2 hr with 1:3000 dilution in skimmed milk. The blot was again washed in the wash buffer 3 times for 1 min each. 12 ml of chemiluminescent substrate (Biorad, Clarity Western ECL substrate, Cat#170-5060) was applied to the blot without shaking in a dark room. The resulting luminescent signal was captured using a camera-based imager.

### Identification of MMP1 Using Mass Spectrometry

The mass spectrometric analysis of MMP1 was performed with a Bruker Ultraflextreme MALDI-TOF mass spectrometer (Bruker Daltonics, Billerica, Mass.) equipped with a frequency tripled Nd:YAG laser operating at 355 nm. Measurements were taken in the positive ion mode with a pulsed ion extraction time of 500 ns in a mass range of 5 to 100 kD. Three replicate spectra were collected for each analysis as 1000 shot composites with a sampling frequency of 1 kHz using automated laser rastering. All analyses were performed with a ferulic acid matrix (20 mg/ml) (Sigma, St. Louis, Mo.) in formic acid (Sigma), acetonitrile (Sigma) and de-ionized water (17%, 33%, 50% v/v). For each sample, 1 μl of matrix was applied to a ground stainless steel sample plate followed by 1 μl of sample and an additional 1 μl of matrix and allowed to air dry. The instrument was calibrated prior to analysis using the ProteoMass Protein MALDI-MS calibration kit (Sigma) with insulin, cytochrome-C, apomyoglobin, aldolase, and albumin size standards. The resulting spectra were baseline smoothed in Bruker Flexanalysis 3.4 software.

SDS PAGE of Type-1 Collagen Degradation by MMP1 without and with Trypsin Inhibitor

The collagen degradation activity of MMP1 was studied using FITC-labeled bovine type-1 collagen (Chondrex, Cat#4001). The Collagen solution was adjusted to pH 7.5 with 1 M NaOH before adding the enzyme. For experiments without trypsin inhibitor, 100 μg of collagen was mixed with 25 μg, 50 μg, 100 μg, and 150 μg of active MMP and incubated for 4 hr at 37° C. The reaction was stopped by adding SDS PAGE loading buffer and 100 mM EDTA. 1 ml of each sample was mixed with 300 μl of the sample buffer containing BME and incubated for 5 min at 95° C. The degradation product was identified by 12% SDS PAGE gels. The gels were excited at 470 nm and the fluorescence image was captured using a CCD imager with filter (Semrock, 600/640 nm). The gel was stained with Coomassie Brilliant Blue R-250 for 15 hr. For experiments with trypsin inhibitor, 100 μl of FITC-labeled collagen (1 mg/ml) was added to 1) 100 μl of protein buffer with 1% Triton® X-100 (control), 2) 50 μl of trypsin at 1 mg/ml and 50 μl of protein buffer with 1% Triton® X-100 (trypsin), 3) 50 μl of trypsin inhibitor (Worthington, Lima Bean, Cat# LS002829) at 1 mg/ml and 50 μl of Triton® X-100 buffer (trypsin+inhibitor), 4/5) 50 μl of either active MMP1 or inactive mutant MMP1 at 1 mg/ml and 50 μl of Triton® X-100 buffer (active or inactive MMP1), 6/7) 50 μl of either active MMP1 or inactive mutant MMP1 at 1 mg/ml and 50 11.1 of trypsin inhibitor (active or inactive MMP1+trypsin inhibitor), and 8) 50 μl of commercial purified MMP1 (Abcam, Cat# ab124850) at 1 mg/ml and 50 μl of Triton® X-100 buffer (commercial MMP1). These eight activity reactions were incubated at 37° C. at 250 rpm for 18 hr and was analyzed using 8% SDS PAGE.

### Zymography of Type-1 Gelatin Degradation by MMP1

The purified MMP1 was analyzed by type-1 gelatin zymography. Type-1 collagen (Type-1 bovine collagen, Nutragen, Advanced Biomatrix, Cat# BA1070-0.5) was converted into type-1 gelatin by heating in a water bath at 65° C. for 30 min. 3 mg/ml gelatin was used to make the 12% zymographic SDS PAGE gel. Different concentrations of purified MMP1 (2 μg to 8 μg) were loaded onto the gel without boiling or heating under non-reducing conditions, i.e., without BME, DDT, and urea. The gel was run at 60 V for 30 min and at 120 V for 1 hr. After running, the gel was rinsed with 100 ml of water three times and incubated in the wash buffer containing 50 mM Tris, 5 mM CaCl₂, 10 μM ZnCl₂, and 2.5% Triton® X-100 for 1 hr to remove SDS. This was followed by 15 hr incubation at 37° C. in the incubating buffer containing 50 mM Tris, 5 mM CaCl₂, and 10 μM ZnCl₂. The gel was stained with Coomassie Brilliant Blue R-250 (Biorad, Cat#161-0436) for 15 hr and analyzed for the activity.

### Breakage of MMP1 Due to Size Exclusion Chromatography

7 ml of trypsin-activated MMP1 was injected into the Sephacryl S-300 column in the AKTA-FPLC (GE) chromatography setup and run at 0.5 ml/min flowrate with the buffer (pH 9) containing 50 mM Tris base, 100 mM NaCl, 200 μM ZnCl₂, and 400 μM CaCl₂. The strong absorption of Triton® X-100 at 280 nm limited the accuracy of the AKTA-FPLC chromatogram. The 60 fractions with 1 ml each were analyzed with the Bradford assay for protein contents. Fractions showing the highest protein contents were analyzed by the SDS PAGE. Chromatography was also performed with a hand packed Sephadex G-75 gravity column.

Determination of k_{cat} for Type-1 Collagen Degradation by MMP1

The rate of collagen degradation per MMP1 per hr, k_{cat}, was calculated using time dependent collagen degradation activity assay. 10 reactions of 500 μl of 1 mg/ml type-1 collagen incubated with 100 μl of 0.5 mg/ml MMP1 were set up in 1.5 ml microcentrifuge tubes at 37° C. and 250 rpm. Every hour, one reaction was centrifuged at 10000 rpm for 10 min using a table top centrifuge (VWR, Galaxy Mini Centrifuge, Model# SN13110667) and precipitated pellet was dried in an oven at 65° C. for 45 min. After drying, the pellets were weighed on an analytical balance (Sartorius, model-ENTRIS 224_15). Untreated collagen was used as control to find the initial weight of the collagen. The weights from each treated sample were subtracted from the control weight to calculate the amount of collagen degraded by MMP1. k_{cat} was calculated by dividing the number of degraded collagen monomers (considering 400 kD molecular weight) by the number of MMP1 (considering 43 kD molecular weight) molecules times the incubation time in hr.

### Determination of Specific Activity of MMP1 Using Synthetic Peptide Substrate

Stock solution of MMP1 at the concentration of 1 ng/μl was made using protein buffer with 1% Triton® X-100. Stock solution of the synthetic MMP1 substrate, MCA-Lys-Pro-Leu-Gly-Leu-DPA-Ala-Arg-NH2, (R&D Systems, Cat# ES010) at the concentration of 20 μM was also made in the same buffer. 50 μl of 1 ng/μ1 MMP1 was loaded into 96-well plate wells and 50 μl of 20 μM substrate was added to each well. To determine the background fluorescence, a blank well with 50 μl of protein buffer and 50 μl of substrate solution was measured without MMP1. Final amounts of MMP1 and substrate per well were 0.05 μg and 10 μM respectively. The 96-well plate was incubated at 37° C. for 1 hr and fluorescence measurements were taken at 1 min intervals in kinetic mode at excitation and emission wavelengths of 360/40 nm and 460/40 nm using a plate reader (Biotek, Synergy2-Cam4, Software-Gen5-1.08). Relative fluorescence was calculated by subtracting fluorescence measurements without the substrate from the measurements with substrate.

Effect of Trypsin and MMP1 on E. coli Proteins

E. coli Rosetta (DE3) PLysS cells with and without recombinant MMP1 plasmid were grown overnight as seed culture and next morning batch culture was set for both strains as described before. 1 g of E. coli cells with and without MMP1 plasmid were reconstituted in lysis buffer (50 mM Tris, 100 mM NaCl, 0.5% Triton® X-100, and pH 9.0) followed by sonication and centrifugation. 100 μl of supernatant was incubated with 100 μg MMP1 and 100 μg trypsin individually and in combination. Protein profiles after degradation were analyzed using 12% resolving SDS PAGE.

Effect of Trypsin and MMP1 on E. coli Proteins with Trypsin Inhibitor

1 g of centrifuged E. coli Rosetta (DE3) pLysS cells containing MMP1 recombinant plasmid was reconstituted in 7 ml of lysis buffer. The cells were disrupted using a sonicator (Branson Digital Sonifier, Model# BBT16031593A) at 40% amplitude for 10 min with a sequence of 10 sec pulse ON and 20 s pulse OFF. Lysate was centrifuged at 11,000 rpm for 15 min. Pellet was discarded and supernatant containing E. coli proteins were stored at 4° C. 100 μl of supernatant was added to 1) 100 μl of protein buffer with 1% Triton® X-100 (control), 2) 50 μl of trypsin at 1 mg/ml and 50 μl of protein buffer with 1% Triton® X-100 (trypsin), 3) 50 μl of trypsin inhibitor (Worthington, Lima Bean, Cat# LS002829) at 1 mg/ml and 50 μl of Triton® X-100 buffer (trypsin+inhibitor), 4/5) 50 μl of either active MMP1 or inactive mutant MMP1 at 1 mg/ml and 50 μl of Triton® X-100 buffer (active or inactive MMP1), 6/7) 50 μl of either active MMP1 or inactive mutant MMP1 at 1 mg/ml and 50 μl of trypsin inhibitor (active or inactive MMP1+trypsin inhibitor), and 8) 50 μl of commercial purified MMP1 (Abcam, Cat# ab124850) at 1 mg/ml and 50 μl of Triton X-100 buffer (commercial MMP1). These eight activity reactions were incubated at 37° C. at 250 rpm for 18 hr and was analyzed using 8% SDS PAGE.

### Results

### SDS-PAGE Analysis of MMP-1 Expression and Activation

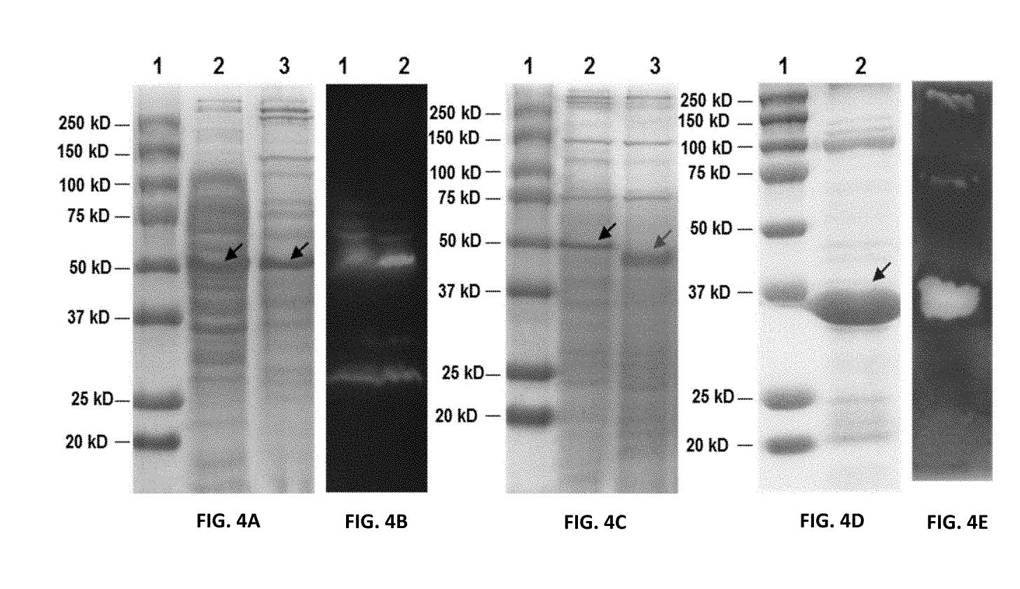

Optimization, expression of MMP-1, and lysis of cells was performed as described above in the Methods section. The resulting bacterial cell lysates were analyzed by SDS-PAGE. The results of this analysis are shown in FIGS. 4A-4E.

FIG. 4A shows SDS PAGE analysis of bacterial cell lysates with (lane 3) and without (lane 2) 1 mM IPTG. Leaky expression of MMP1 was observed without IPTG (lane 2), but MMP1 expression was more prominent with IPTG (lane 3). Western blot of cell lysates using anti-human MMP1 antibody showed autolysis of MMP1 and confirmed leaky expression (FIG. 4B).

Both the supernatant and pellet from centrifuged cell lysates were analyzed using SDS PAGE as shown in FIGS. 4C and 4D, respectively. The supernatant without trypsin treatment showed 54 kD pro-MMP1 (lane 2 of FIG. 4C), indicated by the black arrow), whereas trypsin-treated supernatant showed the 43 kD activated MMP1 (lane 3 of FIG. 4C, indicated by the arrow). The pellet was dissolved in 8 M urea before SDS PAGE and showed a primary band at 28 kD, a fragment containing the catalytic domain of MMP1 (FIG. 4D). Western blot analysis of the pellet using an antibody raised to the catalytic domain of MMP-1 showed that the 28 kDa band in FIG. 4D contains the catalytic domain.

### Purification and Identification of MMP1

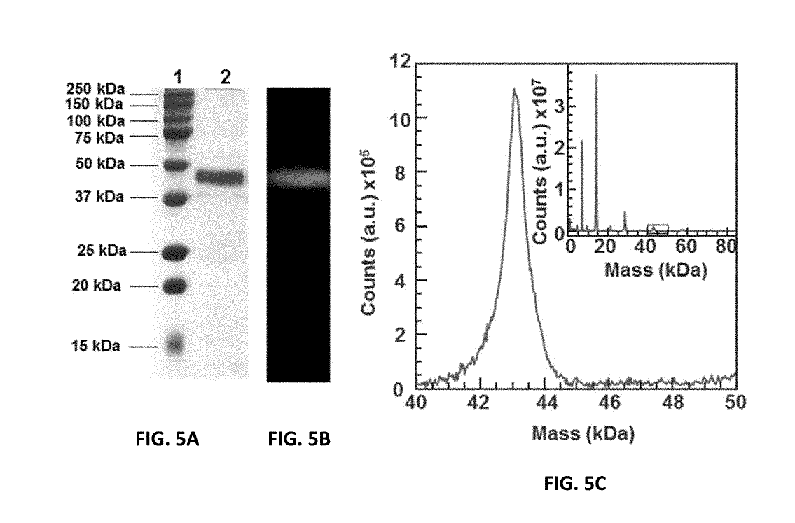

MMP-1 was activated using trypsin treated with Tosyl Phenylalanyl Chloromethyl Ketone (TPCK). Trypsin results in the cleavage of the F—V bond (illustrated in FIG. 3) to produce activated MMP1. Trypsin was used to digest E. coli proteins as well as to activate MMP1 in the supernatant of cell lysates. After trypsin treatment for 15 hr incubation at 37° C., the supernatant was filtered to remove most of the undesired proteins. The resulting MMP1 solution was analyzed using SDS PAGE. Because higher concentrations of Triton® X-100 affect the profiles of SDS PAGE, samples were diluted to 1% Triton® X-100 solution prior to SDS PAGE. The results of this analysis are shown in FIGS. 5A-5C.

FIG. 5A shows the resulting active MMP1. Purified MMP1 was quantified by generating a standard curve with bovine serum albumin (BSA) and Bradford assay. A total of 3.5 mg active MMP1 was obtained in the supernatant at a concentration of 0.5 mg/ml from 1 g bacteria. Western blot confirmed the identity of activated MMP1 (FIG. 5B). The expected mass of activated MMP1 according to ExPASy was 42.5 kD. The mass measured by MALDI-TOF mass spectrometry was 43 kD (FIG. 5C). Fragmentation due to autolysis and/or heat leads to 28 kD MMP1 form that tends to precipitate (FIG. 4D and FIG. 5C inset). Western blot confirmed that both 28 kD (FIG. 4E) and 43 kD forms (FIG. 5B) contain the catalytic domain of MMP1.

### Collagen Degradation Activity of MMP1

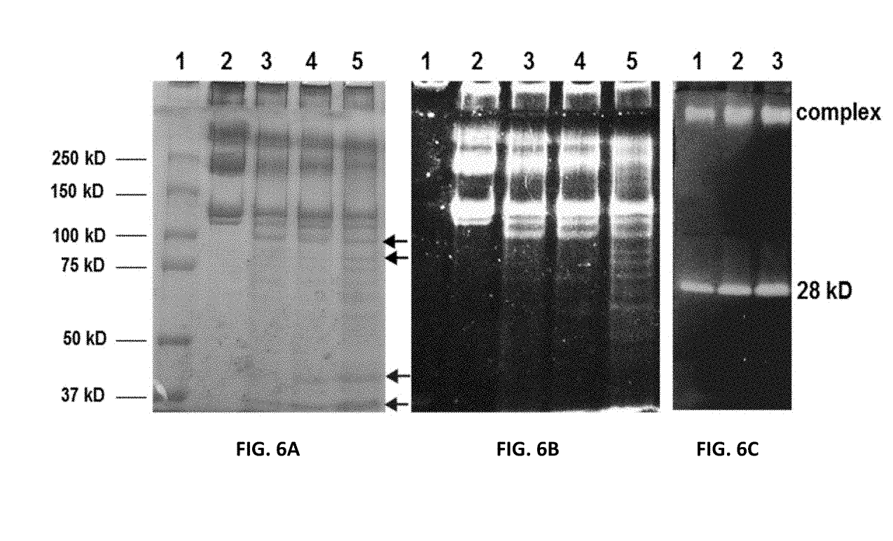

Human MMP1 is known to cleave type-1 collagen into ¾ and ¼ fragments, which can be further degraded by MMP1 itself or other gelatinases. Type-1 collagen monomers are 300 nm in length, 1.5 nm in diameter, and consist of two α(1) and one α(2) chains. To clearly identify cleavage fragments, FITC-labeled bovine type-1 collagen were analyzed by SDS PAGE. As shown in FIGS. 6A and 6B, active MMP1 cleaves type-1 collagen into ¾ and ¼ fragments; intermediate products of collagen cleavage were also prominent as the concentration of MMP1 was increased. This confirmed that the protein was properly folded and remained active in culture supernatants of E. coli. MMP1 activity was also tested by zymography as shown in FIG. 6C, which showed a band at 28 kD and one at a higher molecular weight, but no band was observed at 43 kD most likely because zymography leads to fragmentation of 43 kD MMP1.

MMPs have both specific and non-specific binding sites on collagen monomer and fibril. Additionally, collagen fibrils are insoluble in aqueous buffer making biochemical activity assays difficult. As such, explanation of collagen degradation activity using Michaelis-Menten (MM) kinetics is not straightforward without drastic assumptions because MM kinetics works better when substrate and ligand bind specifically similar to lock-key arrangements. Thus, a weight-based activity assay was developed (see methods) instead of using fluorogenic substrate-based assay. The k_{cat} was calculated to be ˜19 monomers per MMP1 per hour using the weight-based assay with FITC-labeled type-1 (see Table 2), which has the same order of magnitude as the previously reported values.

TABLE 2

| | Weight of | | Weight of | Weight of | | |
|---|---|---|---|---|---|---|
| | empty tube | Weight of tube + | collagen | degraded | k_{cat}/MMP1/hr | k_{cat}/MMP1/hr |
| Sample | W1 | dry pellet W2 | W2 − W1 | collagen | using 1 mg/ml | using 114.2 mg/ml |
| description | (g) | (g) | (g) | (g) | concentration | concentration |
| Collagen + | 1.0006 | 1.0577 | 0.0571 | 0 | NA | NA |
| No MMP1 | | | | | | |
| +MMP1 for 1 hr | 1.0001 | 1.0301 | 0.0300 | 0.0271 | 0.50 | 58 |
| +MMP1 for 2 hr | 0.9908 | 1.0252 | 0.0344 | 0.0227 | 0.21 | 24 |
| +MMP1 for 3 hr | 0.9874 | 1.0146 | 0.0272 | 0.0299 | 0.19 | 22 |
| +MMP1 for 4 hr | 0.9949 | 1.0200 | 0.0251 | 0.0320 | 0.15 | 17 |
| +MMP1 for 5 hr | 0.9988 | 1.0175 | 0.0187 | 0.0384 | 0.14 | 16 |
| +MMP1 for 6 hr | 0.9950 | 1.0167 | 0.0217 | 0.0354 | 0.11 | 13 |
| +MMP1 for 7 hr | 0.9918 | 1.0103 | 0.0185 | 0.0386 | 0.10 | 11 |
| +MMP1 for 8 hr | 1.0055 | 1.0202 | 0.0147 | 0.0424 | 0.10 | 11 |
| +MMP1 for 9 hr | 0.9899 | 1.0023 | 0.0124 | 0.0447 | 0.09 | 10 |
| +MMP1 for 10 hr | 0.9903 | 1.001 | 0.0107 | 0.0464 | 0.09 | 10 |

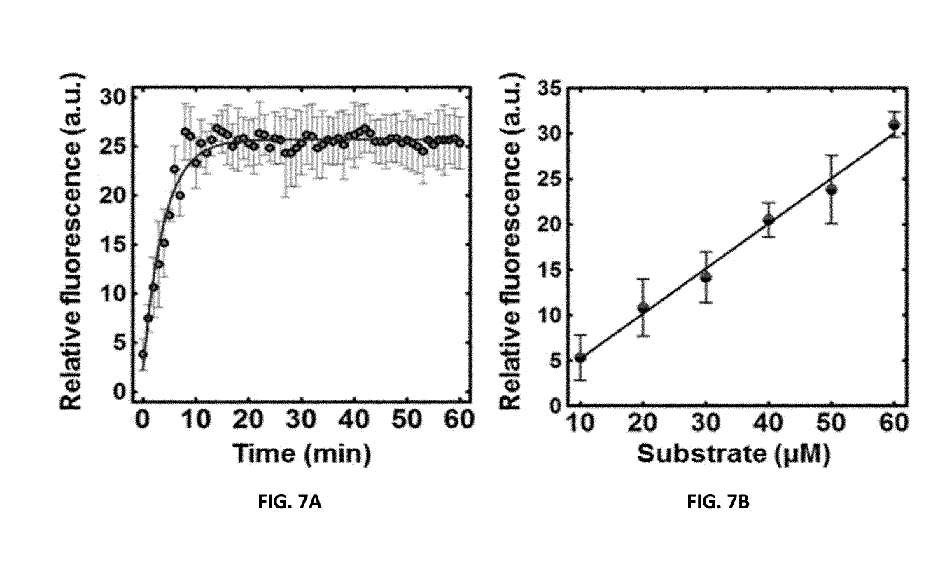

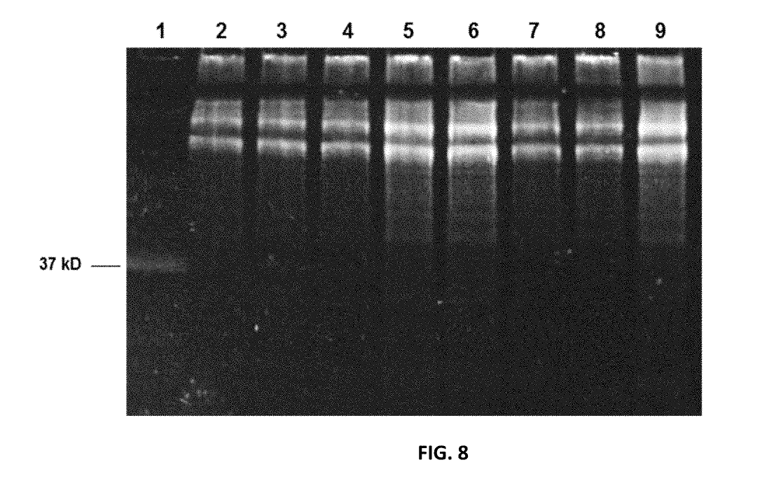

To compare the specific activity of MMP1 purified using the disclosed method, with the previously reported values, a method using a well-known synthetic peptide substrate for MMP1, MCA-Lys-Pro-Leu-Gly-Leu-DPA-Ala-Arg-NH₂ was used. The specific activity was determined to be ˜999 pmol/min/μg at 37° C. (see FIGS. 7A and 7B) as compared to the previously reported value of ˜400 pmol/min/μg for commercially available MMP1 (R&D Systems, Cat#901-MP-010). To confirm that native type-1 collagen was used, the activity of trypsin without (lane 3 of FIG. 8) and with (lane 4 of FIG. 8) trypsin inhibitor was used (trypsin cannot cleave native type-1 collagen). SDS PAGE of collagen degradation with increasing amount of trypsin did not show any degradation. Next, to confirm that the observed collagen degradation activity was due to enzymatic activity of MMP1, a comparison of the catalytically inactive mutant MMP1 without (lane 7, FIG. 8) and with (lane 8 of FIG. 8) trypsin inhibitor was conducted. Both trypsin and catalytically inactive mutant MMP1 (E219Q) with and without trypsin inhibitor did not show degradation of type-1 collagen. Comparison with the activity of commercial MMP1 further confirmed the identity of purified MMP1 (lane 9 of FIG. 8).
Broad-Spectrum Protease Activity of MMP1 on Native E. coli Proteins

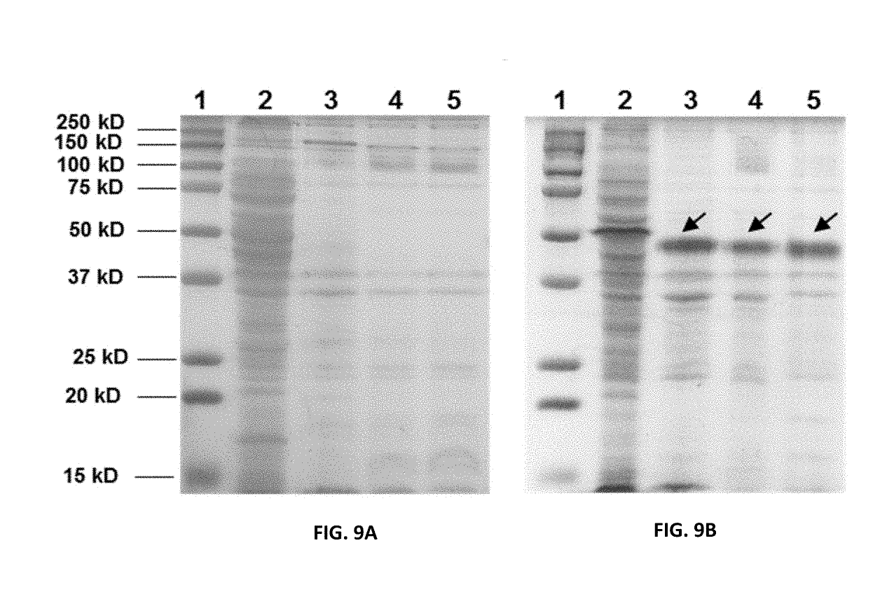

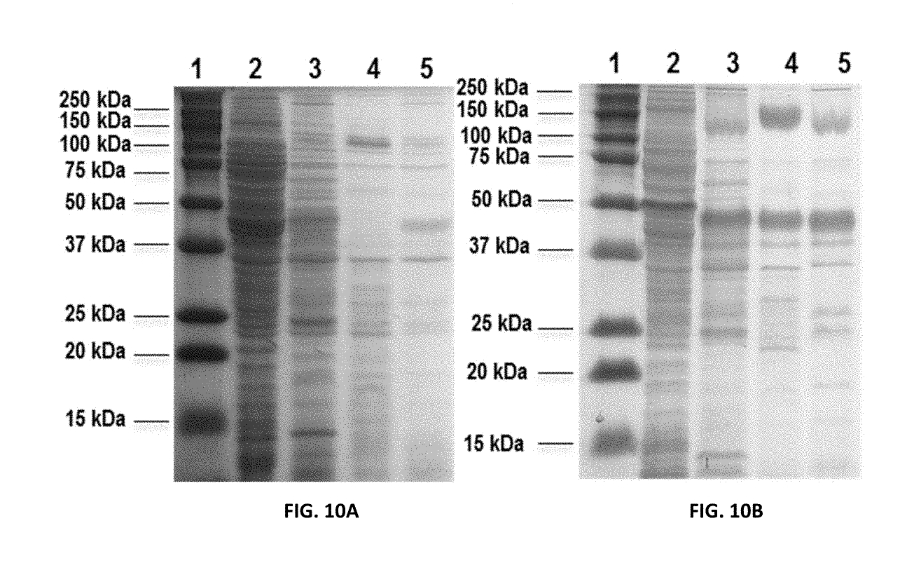

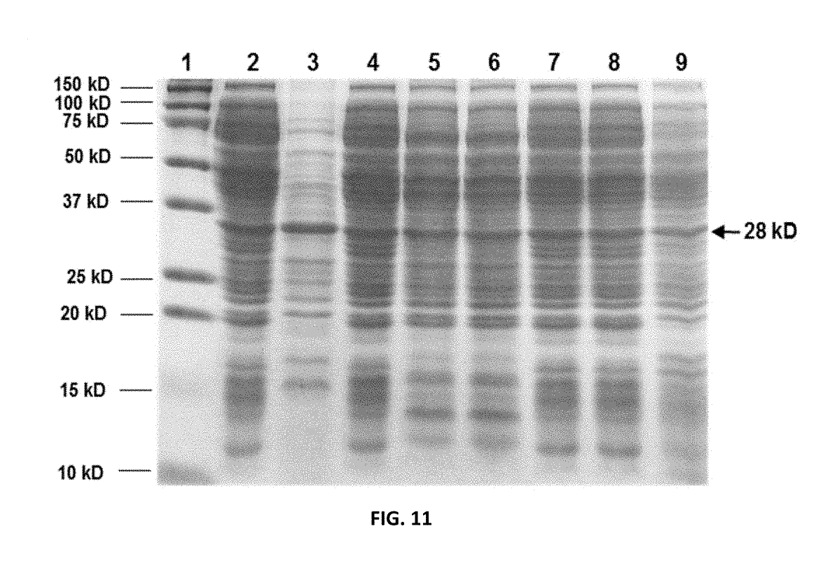

MMPs, in particular MMP1, remodel the extracellular matrix and catalyze a variety of matrix and non-matrix substrates including gelatin, aggrecan, versican, casein, nidogen, serpins, tenasin-C, perlecan, IGFBP-2,3, α1-antichymotrypsin, α1-proteinase inhibitor, pro-MMP1, pro-MMP2, and pro-TNFα. The activity of MMP1 was tested on native proteins produced by Rosetta (DE3) pLysS competent E. coli with and without the MMP1 plasmid. FIG. 9A shows the effect of MMP1 on proteins in the supernatant after cell lysis using lysozyme. Lanes 3, 4, and 5 show significant degradation of native proteins in the supernatant (lane 2) after treatment with MMP1 only, trypsin only, and with both MMP1 and trypsin. FIG. 9B shows the effect of MMP1 on proteins in the supernatant after cell lysis with MMP1 plasmid using lysozyme. FIG. 9B also shows the expression of recombinant 54 kD MMP1 in contrast to FIG. 9A. FIG. 9 shows that both trypsin and active MMP1 digest a significant amount of native E. coli proteins, and that when used simultaneously they digest more of the native proteins. FIG. 9B (lane 3) shows that MMP1 can activate itself through autocatalytic activity. In effect, trypsin is not needed after initial purification of active MMP1. Trypsin can be replaced by activated MMP1, which degrades E. coli proteins and activates pro-MMP1. Cell disruption by sonication produced similar results (FIGS. 10A and 10B). To confirm that the broad-spectrum protease activity of MMP1 was not due to residual trypsin after purification, whole cell lysates of E. coli strains with MMP1 plasmid was treated with different enzymatic conditions. Trypsin, a known broad-spectrum protease, degraded most of the native E. coli proteins (lane 3) as expected, which stopped in the presence of a trypsin inhibitor (lane 4, FIG. 11). MMP1 showed broad-spectrum activity as well to a lesser extent as compared to trypsin. However, MMP1 showed significantly better and complementary degradation of E. coli proteins at ˜11 kD and ˜17 kD (lanes 5, 6, and 9; FIG. 11), which stopped in the presence of inactive mutant MMP1 (lanes 7 and 8; FIG. 11).

The use of freshly prepared Triton® X-100 as a solubilizing agent was found to be beneficial for the solubility, activation, and prevention of complex formation of MMP1. Triton® X-100 is a widely used, relatively inexpensive non-ionic detergent for lysing cells to extract protein and other cellular organelles. Triton® has been known to solubilize membrane proteins in their native state in conjunction with zwitterionic detergents such as CHAPS (3-[(3-cholamido propyl) dimethyl ammonio]-1-propane sulfonate). A related detergent, called CHAPSO, has the same basic chemical structure with an additional hydroxyl functional group. Triton® X-100 was used in the lysis buffer to solubilize MMP1 efficiently, and 0.1 mg/mL trypsin was added to both activate MMP1 and digest E. coli proteins. After 15 hours of incubation and centrifugation, the supernatant was analyzed using 12% SDS-PAGE. As illustrated in FIG. 9A, the activation of MMP1 with trypsin showed a prominent complex of MMP1 at about 100 kDa without Triton® X-100. FIG. 9B illustrates the effect of varying the concentration of Triton® X-100. At 3% Triton® X-100, the MMP1 complex was not noticeable in the SDS-PAGE; by comparison, the use of chymotrypsin gave similar results, but the size of the activated MMP1 was larger than 43 kDa, suggesting an incomplete activation. It should be noted that Triton® X-100 has a strong absorption at 280 nm and therefore may interfere with protein quantification and monitoring of the UV chromatogram in certain applications. Hence, triton compatible Bradford reagent was used to quantify the amount of proteins after purification.

Protein of interest is usually purified using some combination of specific tags, such as the His tag, in combination with column-based liquid chromatography, and MMP1 purification using these methods is known in the art. However, these methods are expensive and low-throughput, which contributes to the high cost of commercial MMP1. Trypsin is an effective protease and has high protease activity; the present inventors have exploited this activity not only to activate the MMP1 but also to degrade the native E. coli proteins present in the culture supernatant. The present inventors also found that trypsin could degrade a significant amount of the E. coli proteins and activate the MMP1 as well. As a result, the active form of human MMP1 can be purified from E. coli using a protease-based method in fewer steps, without using urea, protease inhibitors, specific chromatography columns, or APMA. In less than 72 hours, with minimal reagents and manual effort, it is possible to obtain 3.5 mg of soluble, active MMP1 at 0.5 mg/mL concentration with good purity from 500 mL of E. coli culture (FIG. 11).

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described above is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

We claim:

1. A method of purifying a protein of interest (POI), comprising
a. culturing a recombinant cell comprising an expression vector that comprises a nucleic acid sequence encoding the POI, under conditions suitable for production of the POI;
b. lysing the cells in the presence of a non-ionic detergent to produce a lysate;
c. contacting the soluble portion of the lysate with a protease to which the POI is resistant; and
d. contacting the soluble portion of the lysate with a filter to produce a retentate, and a filtrate containing the POI, thereby purifying the protein of interest.

2. The method of claim 1, wherein protein of interest is a matrix metalloproteinase (MMP).

3. The method of claim 2, wherein the protease is a serine protease.

4. The method of claim 3, wherein the serine protease is trypsin.

5. The method of claim 1, wherein prior to step c), the insoluble portion of the lysate is physically separated from the soluble portion of the lysate.

6. The method of claim 1, wherein the cells are bacterial cells.

7. The method of claim 1, wherein the cells are E. coli cells.

8. The method of claim 1, wherein the nucleic acid sequence is operationally linked to a promoter.

9. The method of claim 8, wherein the promoter is an inducible promoter.

10. The method of claim 8, wherein the promoter is selected from the group consisting of a T7 promoter, a trp promoter, a lac promoter, and an SP6 promoter.

11. The method of claim 1, wherein the expression vector is a bacterial expression vector.

12. The method of claim 11, wherein the expression vector is a pET plasmid.

13. The method of claim 1, wherein the nucleic acid sequence encoding the MMP-1 protein has been modified to increase the amount of MMP-1 protein produced in the recombinant cell.

14. The method of claim 13, wherein modification of the nucleic acid sequence comprises introducing silent, substitution mutations into one or more rare codons in the nucleic acid sequence, thereby eliminating the one or more rare codons from the nucleic acid sequence.

15. The method of claim 1, wherein the non-ionic detergent is a polyoxyethylene.

16. The method of claim 15, wherein the non-ionic detergent is a polysorbate or a non-ionic surfactant.

17. The method of claim 1, wherein the non-ionic detergent is selected from the group consisting of Tween, Triton and Nonidet-P40.

18. The method of claim 1, wherein lysis of the cells comprises sonification, and/or chemical lysis (e.g. lysozyme).

19. The method of claim 1, wherein the filter has a molecular weight cutoff of about 30 kDa.

20. A nucleic acid molecule comprising a nucleic acid at least about 70% identical, at least about 80% identical, at least about 85% identical, at last about 90% identical, at least about 95% identical, at least about 96% identical, at least about 97% identical, at least about 98% identical, at least about 99% identical to SEQ ID NO:1, wherein the difference between the nucleic acid sequence and SEQ ID NO:1 is due, at least in part, to a silent mutation in one or more codons that correspond to one or more rare codons in SEQ ID NO:1 selected from the group consisting of codon 24, codon 49, codon 55, codon 90, codon 91, codon 161, codon 165, codon 202, codon 208, codon 248, codon 259, codon 269, codon 282, codon 287, codon 300, codon 307, codon 337, codon 357, codon 361, codon 372, codon 399, codon 405, codon 412, codon 415, codon 443, codon 453, and codon 467.
